(19) United States
(12) Patent Application Publication
(10) Pub. No.: US 2010/0076316 A1
(43) Pub. Date: Mar. 25, 2010

(54) Ultrasonic probe

(75) Inventor: Yasunobu Hasegawa, Saitama (JP)

Correspondence Address: EDWARDS ANGELL PALMER & DODGE LLP, P.O. BOX 55874, BOSTON, MA, 02205, US

(73) Assignee: NIHON DEMPA KOGYO CO., LTD., Tokyo (JP)

(21) Appl. No.: 12/589,623

(22) Filed: Oct. 26, 2009

Related U.S. Application Data
(63) Continuation-in-part of application No. 11/879,909, filed on Jul. 19, 2007.

(30) Foreign Application Priority Data
Jul. 25, 2006 (JP) 2006-201513

(51) Int. Cl. A61B 8/00

(52) U.S. Cl. 600/459

(57) ABSTRACT

The ultrasonic probe comprises a group of piezoelectric elements including piezoelectric elements arrayed in a long-axis direction thereof; and a rotation mechanism that oscillates the piezoelectric elements to the left and right in a short-axis direction thereof about a center of the long-axis direction. The rotation mechanism has a first bevel gear of a circular-arc shape, a second bevel gear meshing with the first bevel gear and rotating in a horizontal direction, and a drive motor. An optical rotating plate, with the boundary region as a reference, is rotated by less than a predetermined angle, and the boundary region is detected by transmission or shielding of light by the light shielding portion and the light transmission portion, and thereby, based on the detected boundary region, a reference position with respect to an object to be detected of the piezoelectric element group is set.

### CROSS-REFERENCE TO RELATED APPLICATIONS

The present application is a continuation-in-part of co-pending U.S. application Ser. No. 11/879,909 filed Jul. 19, 2007, which is incorporated herein by reference in its entirety.

### BACKGROUND OF THE INVENTION

The present invention relates to an ultrasonic probe in which a group of piezoelectric elements that is a source of ultrasonic waves oscillates in the short-axis direction to obtain a three-dimensional image (hereinafter called a “short-axis oscillating probe”) and, in particular, to a short-axis oscillating probe having a simple configuration in which the operational noise of the probe while operating is minimized to remove that source of discomfort to the patient, and in which a mechanism of detection of a rotation angle of a piezoelectric group is simplified, and detection of a reference position thereof with respect to an object to be detected is facilitated.

1. Field of the Invention

A short-axis oscillating probe that is known in the art obtains a three-dimensional (3D) image by electronically scanning a group of piezoelectric elements in the long-axis direction of the probe and also by mechanically scanning (oscillating) the group of piezoelectric elements in the short-axis direction thereof (see Japanese Unexamined Patent Publication No. 2006-346125 (Patent document 1, prior-art example 1), Japanese Unexamined Patent Publication No. 2003-175033 (Patent document 2, prior-art example 2), and German Patent Publication No. DE3405537A1 (Patent document 3, prior-art example 3).

Since components such as wiring (connective wires) and scan circuitry of this type of short-axis oscillating probe can be configured simply, in comparison with a matrix type of probe in which piezoelectric elements are arrayed horizontally and vertically to provide a two-dimensional electronic scan, this probe can be widely implemented

2. Description of Related Art

A prior-art example 1 of a short-axis oscillating probe is shown in FIG. 5, where FIG. 5A is a section taken along the long-axis direction thereof and FIG. 5B is a section taken along the short-axis direction thereof.

The short-axis oscillating probe of this prior-art example 1 is provided with a group of piezoelectric elements 1 and a rotation mechanism 2, as shown in FIGS. 5A and FIG. 5B. The group of piezoelectric elements 1 is arrayed on a backing member (not to shown in the figure), with the widthwise direction of a plurality of strip shape piezoelectric elements 1a aligned in the long-axis direction and the lengthwise direction thereof aligned in the short-axis direction. The backing member is affixed to the top of a base 4, which is formed in a convex dome shape in the long-axis direction, with the configuration being such that the group of piezoelectric elements 1 is curved outward (convex) in the long-axis direction.

A flexible substrate 5 that has been connected electrically to the group of piezoelectric elements 1 over the entire region of the probe in the long-axis direction thereof is lead out downward from one end side of the probe in the short-axis direction. In this case, a conductive path 5a of the flexible substrate 5 (in FIG. 5A omitted but shown by partially cutaway lines) is connected electrically to a drive electrode (not shown in the figure) of each piezoelectric element 1a. In FIGS. 5A and 5B, the two are connected directly. However, the drive electrode of each piezoelectric element 1a may be connected indirectly to the conductive path 5a by means such as silver foil and conductive wiring.

In the prior-art example 1, the rotation mechanism 2 shown in FIG. 5A comprises a retaining plate 6, a case 7, a first bevel gear 8a, a second bevel gear 8b, a rotation shaft 9, and a drive motor 10 that has been attached to a framing member 7b. The retaining plate 6 has leg portions 6a and 6b on the lower surface thereof on both edge sides in the long-axis direction, and the base 4 supporting the group of piezoelectric elements 1 is affixed to the upper surface thereof. Center shafts 11a and 11b that penetrate through the corresponding leg portions 6a and 6b are provided in the long-axis direction (on the line X-X in the horizontal direction shown in FIG. 5A), on bearings 11c and 11d. The leg portions 6a and 6b are provided to be freely rotatable with respect to the center shafts 11a and 11b.

Furthermore, the case 7 is formed to be concave in section with the upper surface thereof being open, and projecting ends of the center shafts 11a and 11b that protrude from the leg portions 6a and 6b are connected (affixed) to peripheral walls of the case 7. A slit 12 (see FIG. 5B) is formed in the long-axis direction in the bottom wall of the case 7, and the flexible substrate 5 from the group of piezoelectric elements 1 is lead out to the exterior of the framing member 7b therethrough. A material such as a synthetic resin 13 is embedded in the slit 12 to seal the same.

The first bevel gear 8a is provided on the inner surface of the leg portion 6a, below the center shafts 11a and 11b, and has teeth in a circular-arc shape (a fan shape) with a peak thereof at the lower end in the vertical direction. The second bevel gear 8b is borne on the tip end side of the rotation shaft 9, which is in the vertical direction perpendicular to the center shafts 11a and 11b (the line X-X), and engages with the first bevel gear 8a to rotate in the horizontal direction (the X-X direction). The rotation shaft 9 is lead out from the bottom wall of the case 7 to outside of the case 7, and is sealed by a seal ring 14a, and the other end thereof is gear coupled (meshed) with the drive motor 10 using for example metal spur gears 15a and 15b.

In this prior-art example 1, the first bevel gear 8a and the second bevel gear 8b are made of metal, and the diameter of the equivalent circle of the circular-arc-shaped teeth of the first bevel gear 8a is greater than the diameter of the second bevel gear 8b. In addition, the diameter of the metal gear 15a affixed to the rotation shaft 9 is greater than the diameter of the metal gear 15b of the drive motor 10.

By making the gear ratio from the drive motor 10 to the first bevel gear 8a greater in this manner, this configuration ensures that the rotational force (torque) of the drive motor 10 is increased and maintained, in driving and transmitting the rotational force to the first bevel gear 8a. A cover (see reference symbol 7a in FIG. 1) that encloses the group of piezoelectric elements 1 is provided for the case 7, the group of piezoelectric elements 1 and other components are hermetically sealed therein, and the interior of the case 7 that is sealed with the cover is filled with an ultrasound transmission medium such as oil.

In the thus-configured prior-art example 1, due to the oscillation and rotation of the motor 10 that rotates (oscillates) the second bevel gear 8b that configures the rotation mechanism 2, horizontally to left and right, the first bevel gear 8a oscillates with respect to the vertical plane upwards and inclined to the left or right with the peak thereof as the center. In other words, the first bevel gear 8a rotates and oscillates by for example 70° to the left and right of the vertical direction with the peak as the center. Thus the leg portions 6a and 6b of the retaining plate 6 rotate and oscillate to the left and right with respect to the center shafts 11a and 11b, while the group of piezoelectric elements 1 rotate and oscillate to the left and right in the short-axis direction, in the opposite direction to the first bevel gear 8a.

Furthermore, in prior-art example 2, the drive motor and the rotation shaft on the piezoelectric element side are directly driven by a pulley linkage using a belt.

Furthermore, in prior-art example 3, in the ultrasonic probe, the rotation shaft is rotated by the two drive motors, by a pulley linkage using a belt, and a metal first bevel gear is fixed to the tip end of the rotation shaft, and by means of this first bevel gear, a metal second bevel gear meshed therewith is rotated, to thereby rotate and oscillate the ultrasonic oscillating head that is fixed to the tip end of the second bevel gear.

However, in the short-axis oscillating probe of the prior-art 1 configured as described above, since the first bevel gear 8a and the second bevel gear 8b both made of metal are used, when these metal gears engage, they emit characteristic metallic noises, which raises a problem in that it causes discomfort to the doctor (operator) and, in particular, to the patient, during operation.

In the abovementioned prior-art example 2, the generation of metallic noise is removed, because the motor and the rotation shaft on the piezoelectric element side are directly driven by a pulley linkage using a belt. However, in the prior-art example 2, there is no two-stage linkage using bevel gears in addition to the gear that is connected directly to the drive motor, as described above, so it is necessary to increase the diameter ratio of the pulleys to ensure that the rotational force of the motor is transferred reliably to the rotation shaft on the piezoelectric element side. Since the pulley on the piezoelectric element side is thus increased in size, it is difficult to design a miniaturized compact probe.

Furthermore, in prior-art 3, the rotation shaft is rotated by a pulley linkage using a belt, and a pair of metal bevel gears that are meshed with each other are used to rotate and oscillate the piezoelectric drive head. Therefore, there is the problem in that a metallic noise occurs at the time of meshing and rotating of the pair of metal gears.

An objective of the present invention is to provide a short-axis oscillating probe in which the generation of metallic noise by the meshing of gears is suppressed, making it possible to remove a source of discomfort to the operator and the patient, and in which the setting of a reference position of a piezoelectric group with respect to an object to be detected is facilitated.

### SUMMARY OF THE INVENTION

The present invention relates to an ultrasonic probe including: a group of piezoelectric elements consisting of a plurality of piezoelectric elements of a narrow strip shape arrayed in a long-axis direction thereof; and a rotation mechanism that oscillates the group of piezoelectric elements to the left and right in a short-axis direction thereof about a center of the long-axis direction; wherein the rotation mechanism is provided with a first bevel gear of a circular-arc shape in plan view, a second bevel gear meshing with the first bevel gear and rotating in a horizontal direction, a drive motor that rotates the second bevel gear via a rotation shaft, and a reference position detecting sensor that detects a reference position of a short axis direction of the piezoelectric element group; the construction being such that; the rotation shaft of the second bevel gear and the rotation shaft of the drive motor are linked by a pulley linkage using a timing belt; and also the first bevel gear is formed from a synthetic resin, and an optical rotating plate of the reference position detecting sensor that has a boundary region between a light shielding portion and a light transmission portion, is coupled to the rotation shaft, and the light shielding portion and the light transmission portion are formed in sequence with the boundary region as a reference, at a predetermined angle in opposite directions to each other from the center of the optical rotating plate, and also the optical rotating plate, with the boundary region as a reference, is rotated by less than the predetermined angle, and the boundary region is detected by transmission or shielding of light by the light shielding portion and the light transmission portion, and thereby, based on the detected boundary region, a reference position with respect to an object to be detected of the piezoelectric element group is set.

Since in this configuration there is a pulley linkage using a belt between the rotation shaft of the second bevel gear and the drive motor, and the first bevel gear is made of a synthetic resin, there is no engagement between metal gears and thus there is no generation of the characteristic metallic noise during rotation. This makes it possible to remove a source of discomfort to the operator of the ultrasonic probe and, in particular, to the patient. Moreover, setting of the reference position with respect to the object to be detected of the piezoelectric element group can be facilitated, and hence the diagnosis burden on the operator is considerably reduced.

### BRIEF DESCRIPTION OF THE DRAWINGS

FIG. 1 is a drawing for explaining an embodiment of a short-axis oscillating probe of the present invention, wherein FIG. 1A is a longitudinal section in a long axis direction and FIG. 1B is a transverse section in a short axis direction.

FIG. 2 is a perspective view of the short-axis oscillating probe of the present invention shown in FIG. 1.

FIG. 3 is a perspective view of a reference position sensor of the short-axis oscillating probe of the present invention shown in FIG. 1.

FIG. 4 is a plan view showing a rotation position of an optical rotating plate of the reference position sensor, for explaining an operation of the reference position sensor shown in FIG. 3, wherein FIG. 4A shows a state where a reference position P of the optical rotating plate is rotated counterclockwise by θ2, and FIG. 4B shows a state where the reference position P is rotated clockwise by θ1.

FIG. 5 is a drawing for explaining a conventional short-axis oscillating probe, wherein FIG. 5A is a cross-section in a long axis direction and FIG. 5B is a transverse section in the same short axis direction.

### DETAILED DESCRIPTION OF PREFERRED EMBODIMENT

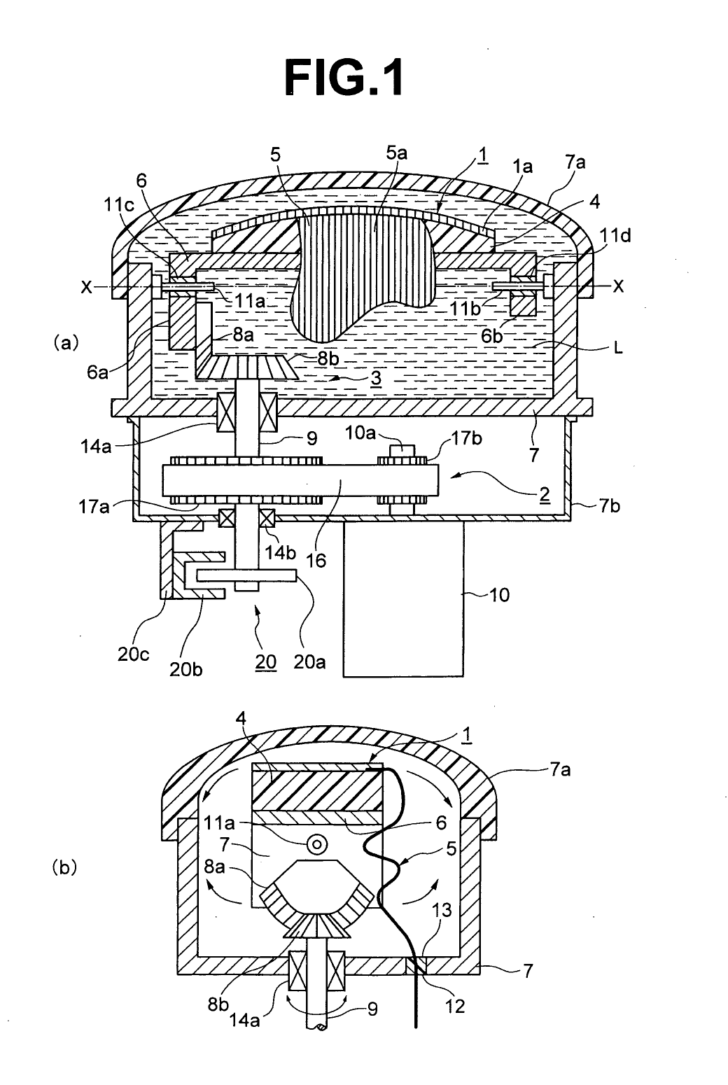

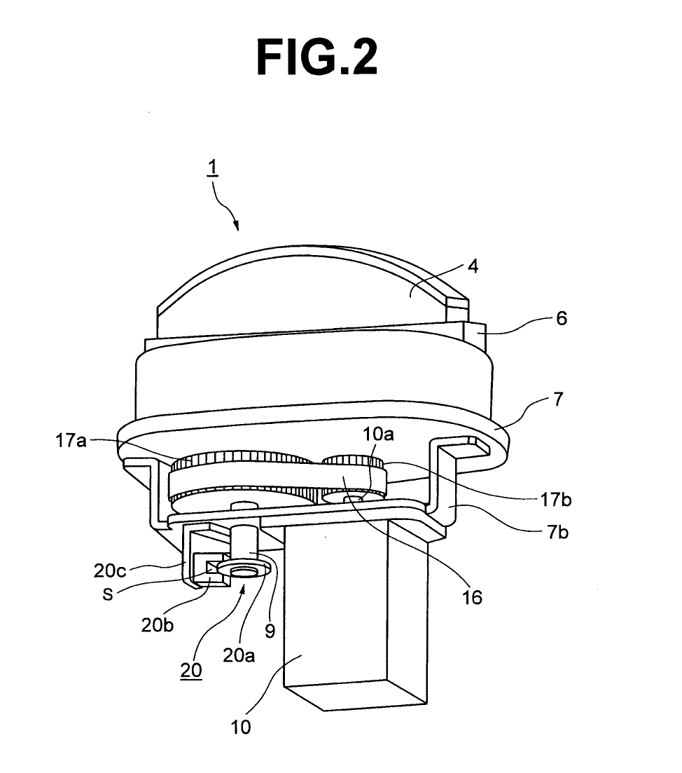

FIG. 1A is a drawing for explaining an embodiment of a short-axis oscillating probe of the present invention, being a cross-section in the long axis direction. FIG. 1B is a transverse section in the short axis direction, and FIG. 2 is a perspective view thereof.

The short-axis oscillating probe of the present invention is provided with a group of piezoelectric elements 1, and a rotation mechanism 2 thereof, and a reference position detection sensor 20.

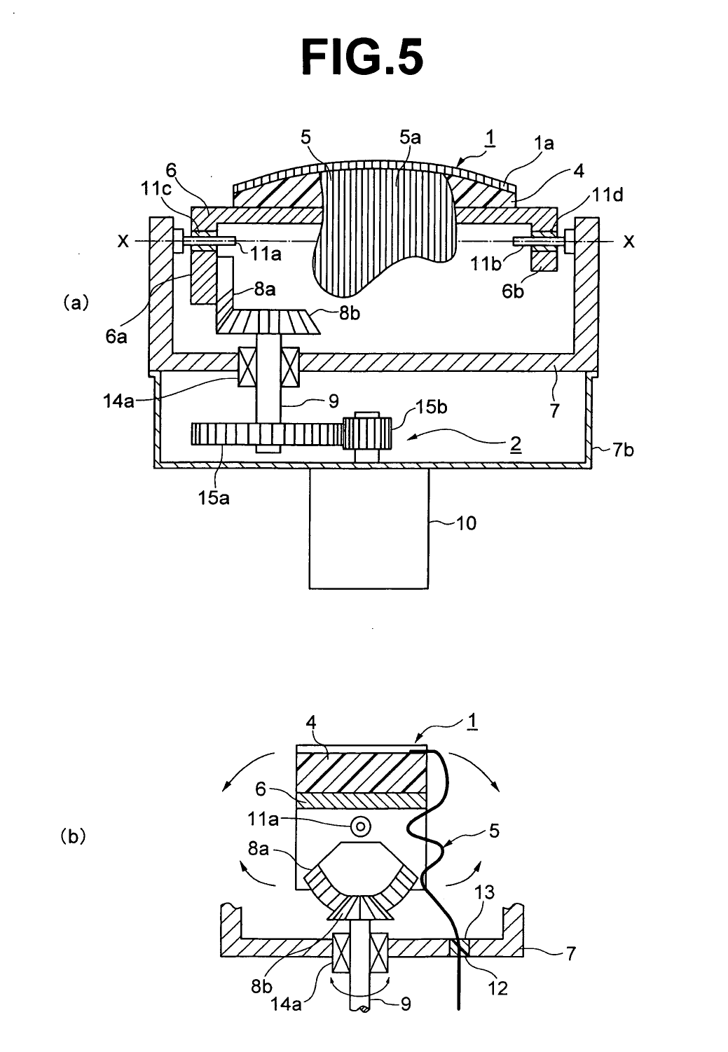

The group of piezoelectric elements 1 are configured in a convex form side by side on a backing member (not shown in the figure) that is attached to an upper surface of a base 4, with the widthwise direction of a large number of piezoelectric elements 1a in the long-axis direction. A flexible substrate 5 that is connected electrically to the group of piezoelectric elements 1 through a conductive path 5a (in FIG. 5A omitted but shown by partially cutaway lines) is lead out from one end side in the short-axis direction of the probe through a slit 12 embedded with synthetic resin 13 and thus sealed.

The rotation mechanism 2 includes: a retaining plate 6 with the group of piezoelectric elements 1 affixed to the upper surface thereof a case 7 of an indented shape and having center shafts 11a and 11b, which rotate freely in leg portions 6a and 6b on both sides of the retaining plate 6 and engage smoothly with bearings 11c and 11d; a first bevel gear 8a, which is provided beneath one leg portion 6a of the retaining plate 6 and which has a circular-arc shape (fan shape) in plan view; a second bevel gear 8b, which meshes with the first bevel gear 8a and which has a rotation shaft 9 that is sealed via a seal ring 14a, and that is sealed and lead out from a bottom wall of the case 7 to the outside of a framing member 7b; and a drive motor (stepping motor) 10 attached to the framing member 7b.

Here, the fan shape first bevel gear 8a on the driven side is formed of a synthetic resin. Moreover, the second bevel gear 8b on the drive side which is subjected to driving torque and hence requires appropriate mechanical strength, is made of metal. In addition, the rotation shaft 9 of the second bevel gear 8b and a rotation shaft 10a of the drive motor (stepping motor) 10 are linked by a timing pulley using a belt 16.

Here, for the material for the synthetic resin first bevel gear 8a, polyacetal resin (abbreviated to POM) is used. In POM resin, amorphous parts and crystalline parts coexist. Therefore, it is an excellent engineering plastic in strength, rigidity, impact resistance, sliding performance, and so on. It is light weight compared to the metal bevel gear, and is extremely good in oil resistance and fabrication ability.

The probe of this invention is used with an ultrasound transmission medium such as oil filled into the case 7 that is sealed by a cover 7a. Therefore, the first bevel gear 8a made of POM plastic having excellent oil resistance, is ideal as the driving member of this type of probe.

Here, the diameter of the first bevel gear 8a is larger than that of the second bevel gear 8b, and a pulley 17a on the rotation shaft 9 is larger than a pulley 17b of the drive motor 10 (the gear ratio thereof is larger), and thus the rotational force of the drive motor 10 is increased. Moreover, mutually meshing timing pulleys and a timing belt are used in which concave-convex grooves are provided in the outer periphery of the pulleys 17a and 17b and in the face of the timing belt 16 that meshes with the pulleys 17a and 17b, so that driving force is transmitted reliably from the pulley 17b to the pulley 17a. Here in the timing pulley and belt mechanism that is a first stage drive device, the reduction ratio is for example 2.8:1, and in the bevel gear drive mechanism that is a second stage drive device, the reduction ratio is for example 2.1:1. Therefore sufficient speed reduction can be obtained by combining the two drive mechanisms.

Since this configuration ensures that the connection between the rotation shaft 9 of the second bevel gear 8b and the rotation shaft 10a of the drive motor 10 is by a pulley linkage using the timing belt 16 and the timing pulleys 17a and 17b, the driving force can be transferred reliably and there is no metallic objectionable noise generated by the meshing of the aforementioned metal gears 15a and 15b shown for example in FIG. 5A, as in the prior-art example. In addition, since the first bevel gear 8a is formed of a synthetic resin and the second bevel gear 8b is made of metal, any metallic noise generated when those gears engage can be minimized. Thus the generation of metallic noise during operation of the probe is suppressed, thereby making it possible to remove a source of discomfort to the operator and, in particular, to the patient.

That is to say, in the probe of the present invention, due to the oscillation and rotation of the motor 10 that rotates (oscillates) the second bevel gear 8b that configures the rotation mechanism 2, horizontally to left and right, this oscillation and rotation is transmitted from the pulley 17b through the timing belt 16 to the pulley 17a, so that the rotation shaft 9 oscillates causing the first bevel gear 8a to oscillate with respect to the vertical plane upward and inclined to the left or right with the peak thereof as the center. In other words, the first bevel gear 8a rotates and oscillates by for example 75° (excluding an acceleration and deceleration range during left/right oscillation) to the left and right of the vertical direction with the peak as the center. Thus the leg portions 6a and 6b of the retaining plate 6 rotate and oscillate to the left and right with respect to the center shafts 11a and 11b, while the group of piezoelectric elements 1 rotate and oscillate to the left and right in the short-axis direction, in the opposite direction to the first bevel gear 8a. Furthermore, the rotation angle in the short axis direction of the piezoelectric group 1 is detected from the reference position by a later described reference position detection sensor of the rotation shaft 9, and living information from the object to be detected (organism) is accurately obtained.

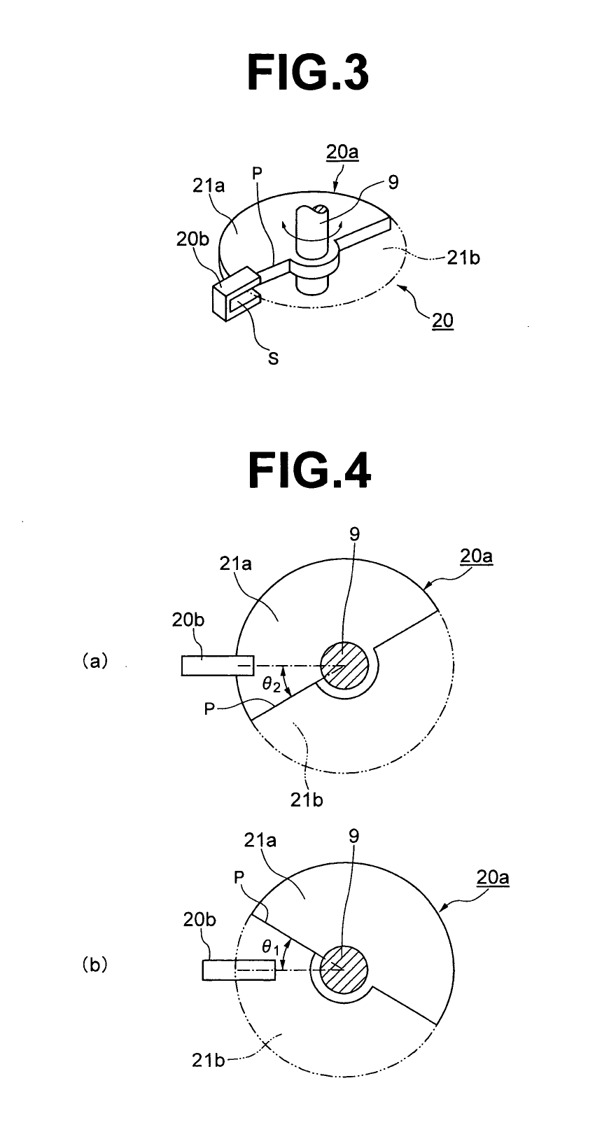

Moreover, the short axis oscillation probe of the present invention, is characterized in that there is provided a reference position detection sensor 20 as shown in FIG. 3.

That is to say, the reference position detection sensor 20, as shown in FIG. 3, comprises an optical rotating plate 21a that is connected by a screw or the like integrally to the rotation shaft 9 that drives the first bevel gear 8a via the second bevel gear 8b, and a photodetector 20b that is attached by a screw or the like to the bottom underside of the framing member 7b as shown in FIG. 1, having a light emission and reception portion of a C shape in cross-section.

The optical rotating plate 21a is a semi-circle (half moon) shape in plan view, made up of a light shielding section 21a and a light transmission section 21b (the region shown by the chain line in FIG. 3), and has a boundary region P comprising a straight line corner between the two. The light shielding section 21a and the light transmission section 21b are formed in sequence with the boundary region P as a reference, spaced 180° apart in opposite directions to each other from a rotation center of the optical rotating plate 21a.

Furthermore, rotation and oscillation of the optical rotating plate 21a shown in FIG. 3, is limited to an angle range of less than 180° in mutually opposite directions, with the boundary region P as a reference. Here, the rotation thereof is limited to within an angle of 90° in mutually opposite directions. This rotation limit depends on the rotation of the rotation shaft 9 that is gear connected to the drive motor 10 shown in FIG. 1. Furthermore, the photodetector 20b is fixed to a framing member 20c shown in FIG. 1A by a retainer such as a screw via a shim or the like, and the outer peripheral portion of the optical rotating plate 21a is positioned to rotate in a shaped space S (C shape in cross-section) of the photodetector 20b.

Here, the initial position of the optical rotating plate 21a (reference position) is a position where the boundary region P is positioned in the center of the C shape space S of the photodetector 20b, which is the switching point (on or off) for transmission or non transmission of the light in the light emission and reception space of the photodetector 20b. In this case, in the piezoelectric group 1 shown in FIG. 1, the center line that bisects the short axis direction from the rotation center is arranged at the reference position matching with the center of the cover 7a shown in FIG. 1, that is, the central top face.

In such a short axis probe of the present invention, operation of the short axis oscillation element is started by pressing a start button (not shown in the figure). Here, before starting operation of the probe, for example as shown in FIG. 4A, the boundary region P of the optical rotating plate 20a is rotated within 90° (θ2) in the counterclockwise direction from the reference position, so that the light shielding section 21a is positioned in the space portion S of the photodetector 20b. In this case, by pressing the start button, at first, the photodetector 20b catches the presence of the light shielding section 21a, and detects a shield signal. Then, based on the shield signal, the drive motor 10 is driven, and the optical rotating plate 20a is rotated clockwise. Next, the boundary region P of the optical rotating plate 20a which becomes the boundary for shielding or transmission of light, is detected. Then the drive motor 10 is stopped to set the reference position.

Furthermore, before starting operation of the probe, as shown in FIG. 4B, the boundary region P of the optical rotating plate 21a is rotated within 90° (θ1) in the clockwise direction from the reference position, and the light transmission section 21b is positioned in the space portion S of the photodetector 20b. In this case, by pressing the start button, at first, the photodetector 20b detects the transmission signal. Then, based on the detected transmission signal, the drive motor 10 is driven, and the optical rotating plate 20a is rotated counterclockwise. Next, similarly to before, the boundary region P is set to the reference position.

By means of these operations, the group of piezoelectric elements 1 is matched with the center of the cover 7a and set to the reference position of the central front face. Moreover, the rotation mechanism 3 linked to the drive motor 10 and the rotation shaft 9 shown in FIG. 1 causes the group of piezoelectric elements 1 to rotate and oscillate left and right from the reference position so that ultrasound is transmitted and received with respect to the object to be detected. Then, a three dimensional (3D) image of the object to be detected can be obtained from a previously set relation between the number of positive and negative pulses and the rotation angle. For example, when the pulse is positive, the group of piezoelectric elements 1 is rotated to the left, and when negative, it is rotated to the right.

That is, according to this configuration, the optical rotating plate 21a is rotated and oscillated, limited to within 90° in mutually opposite directions with the boundary region P as a reference. Consequently, the photodetector 20b detects the shielding signal or the transmission signal that depends on the rotation position of the optical rotating plate 21a, and when the optical rotating plate 21a is rotated in the clockwise of counterclockwise direction according to a previous setting, only the boundary region P on one end side is present within the rotation angle. Consequently, the boundary region P can be reliably detected, and setting of the reference position of the group of piezoelectric elements 1 can be performed easily with a simple mechanism.

Consequently, the reference position of the group of piezoelectric elements 1 can be set accurately, and living body information of an object to be detected can be obtained from an accurate position.

What is claimed is:

1. An ultrasonic probe including: a group of piezoelectric elements comprising of a plurality of piezoelectric elements of a narrow strip shape arrayed in a long-axis direction thereof; and a rotation mechanism that oscillates said group of piezoelectric elements to the left and right in a short-axis direction thereof about a center of said long-axis direction; wherein said rotation mechanism is provided with a first bevel gear of a circular-arc shape in plan view, a second bevel gear meshing with said first bevel gear and rotating in a horizontal direction, a drive motor that rotates said second bevel gear via a rotation shaft, and a reference position detecting sensor that detects a reference position of a short axis direction of said piezoelectric element group; wherein the rotation shaft of said second bevel gear and the rotation shaft of said drive motor are linked by a pulley linkage using a timing belt; and also said first bevel gear is formed from a synthetic resin, and said second bevel gear is made of metal, and an optical rotating plate of said reference position detecting sensor that has a boundary region between a light shielding portion and a light transmission portion, is coupled to said rotation shaft, and said light shielding portion and said light transmission portion are formed in sequence with said boundary region as a reference, at a predetermined angle in opposite directions to each other from the center of said optical rotating plate, and also said optical rotating plate, with said boundary region as a reference, is rotated by less than said predetermined angle, and said boundary region is detected by transmission or shielding of light by said light shielding portion and said light transmission portion, and thereby, based on the detected boundary region, a reference position with respect to an object to be detected of said piezoelectric element group is set.
